(19) United States
(12) Patent Application Publication
(10) Pub. No.: US 2007/0059368 A1
(43) Pub. Date: Mar. 15, 2007

(54) Modified release formulations of anti-irritability drugs

(76) Inventors: S. Rao Cherukuri, Vienna, VA (US); Vittorino Ravelli, Milano (IT)

Correspondence Address: THORPE NORTH & WESTERN, LLP., 8180 SOUTH 700 EAST, SUITE 200, SANDY, UT, 84070, US

(21) Appl. No.: 11/443,665

(22) Filed: May 30, 2006

Related U.S. Application Data
(60) Provisional application No. 60/686,005, filed on May 31, 2005.

(51) Int. Cl. A61K 9/26

(52) U.S. Cl. 424/469

(57) ABSTRACT

Modified or extended release formulations containing mesalamine compounds and associated methods are disclosed and described. In some aspects, such formulations may be substantially bioequivalent to known FDA approved mesalamine formulations such as PENTASA®.

### PRIORITY DATA

This application claims priority to U.S. Provisional Patent Application Ser. No. 60/686,005, filed May 31, 2005, which is incorporated herein by reference.

### FIELD OF THE INVENTION

The present invention relates to mesalamine compound containing formulations with desired in-vitro and in-vivo characteristics and associated methods which are simple to formulate and economical to manufacture on a commercial scale. Accordingly, the present invention involves the field of pharmaceutical sciences.

### BACKGROUND OF THE INVENTION

Modified release mesalamine formulations are desirable because they are expected to provide prolonged and some times more site-specific therapeutic benefits in the treatment of disorders such as irritable bowel syndrome, Crohn's disease, etc. Examples of various known modified release mesalamine formulations may be found in U.S. Pat. Nos. 5,811,388; 6,004,581; and 4,980,173, each of which are incorporated herein by reference.

While mesalamine has been used for many years as an active agent to treat the foregoing conditions, there has been, to date, no generic mesalamine product on the market that is approved by the FDA as being pharmaceutically equivalent to known brand products ASACOL® or PENTASA®. One reason appears to be the interindividual variability among patients in their physiological make-up which causes deviations in gastric motility and the resultant drug release and absorption. Consequently, there has been great difficulty in devising a modified release mesalamine dosage form that provides desirable in vivo drug release. Perhaps another factor is the complexity of the prior art disclosures in terms of their formulation and manufacturing steps.

Accordingly, there is an undisputed commercial need for modified mesalamine dosage form that is pharmaceutically equivalent to the FDA-approved brand products PENTASA® or ASCOL®.

### SUMMARY OF THE INVENTION

Methods are provided for formulating and manufacturing modified release mesalamine dosage forms for oral delivery. Also provided herein are dosage forms thus produced. Methods are also provided for administering such modified dosage forms to a mammal such as humans and members of the animal kingdom. In some aspects, the dosage form is a capsule. In some aspects, the dosage form is a tablet. The amount of mesalamine per dosage form can be, as stated conventionally, from about 200 mg to about 800 mg, including specific intermediate amounts such as 250 mg, 300 mg, 400 mg, 500 mg, 600 mg and 750 mg.

These dosage forms provide a dissolution profile such that: about 15% to about 25% of the drug is released by 60 minutes; about 35% to about 45% of the drug is released by 2 hrs; about 70% to about 85% of the drug is released by 4 hrs; and about 95% to about 105% of the drug is released by 8 hrs when dissolution test is performed using pH 7.5 phosphate buffer.

Alternatively, these dosage forms provide a dissolution profile such that: about 15% or less of the drug is released by 60 minutes; about 20% to about 35% of the drug is released by 2 hrs; about 40% to about 60% of the drug is released by 4 hrs; and about 75% to about 90% of the drug is released by 8 hrs when dissolution test is performed using pH 6.8 phosphate buffer and simulated intestinal fluid without pancreatin.

In yet another aspect, these dosage forms provide a dissolution profile such that: about 20% to about 45% of the drug is released by 60 minutes; about 35% to about 75% of the drug is released by 2 hrs; about 90% to about 100% of the drug is released by 4 hrs, when dissolution test is performed using pH 1.2 simulated gastric fluid without pepsin.

In one other aspect, these dosage forms provide a dissolution profile such that: about 3% to about 6% of the drug is released by 60 minutes; about 8% to about 12% of the drug is released by 2 hrs; about 16% to about 20% of the drug is released by 4 hrs; and more than about 25% the drug is released by 8 hrs when dissolution test is performed using pH 4.5 phosphate buffer.

The dosage forms may be used to treat irritable bowel syndrome, Crohn's disease, among others.

In one aspect, the method comprises the following steps:
a) preparing a mixture of mesalamine and one or more pharmaceutically acceptable excipients to form a mesalamine-excipient mixture;
b) granulate the mesalamine-excipient mixture in the presence of a water-impermeable polymer to produce mesalamine granulates;
c) spheronize and extrude the mesalamine granulates to produce mesalamine cores, and optionally drying and sieving said cores;
d) prepare a dispersion of a water-impermeable polymer and a water-swellable polymer to produce a coating polymer dispersion;
e) coat said mesalamine cores with said coating polymer dispersion to obtain coated mesalamine cores; and
f) provide modified release mesalamine capsules by filling empty capsules with coated mesalamine cores.

In another aspect, the method of making a modified release mesalamine oral dosage form comprises:
a) providing an inert core of substantially uniform size;
b) providing mesalamine dispersion and optionally a binder dispersion;
c) layering said core with mesalamine dispersion simultaneously with or after optional layering of said core with binder dispersion to provide mesalamine core;
d) preparing a dispersion of a water-impermeable polymer and a water-swellable polymer to produce a coating polymer dispersion;
e) coating said mesalamine core with said coating polymer dispersion to obtain coated mesalamine core; and
f) providing modified release mesalamine capsules by filling empty capsules with one or more coated mesalamine cores
In another aspect, the method of making a modified release mesalamine oral dosage form comprises:
a) providing an inert core of substantially uniform size;
b) providing mesalamine dispersion and optionally a binder dispersion
c) layering said core with mesalamine dispersion simultaneously with or after optional layering of said core with binder dispersion to provide a mesalamine core;
d) preparing a dispersion of a water-impermeable polymer and a water-swellable polymer to produce a coating polymer dispersion;
e) coating said mesalamine core with said coating polymer dispersion to obtain coated mesalamine core; and
f) providing modified release mesalamine tablets by compressing one or more said mesalamine coated cores together with optional pharmaceutically acceptable excipients.

In one aspect, the one or more pharmaceutically acceptable exicipients may be selected from the group consisting of: microcrystalline cellulose, dibasic calcium phosphate dihydrate, starch, sodium starch glycolate, crospovidone, croscarmellose sodium, magnesium stearate, lactose, maleic acid, colloidal silicon dioxide, talc, and glyceryl behenate, or a mixture thereof.

In another aspect, the water-impermeable polymer is selected from the group consisting of ethylcellulose, propylcellulose, isopropylcellulose, or a mixture thereof.

In another aspect, the water-swellable polymer is selected from the group consisting of methylcellulose (MC), carboxymethylcellulose (CMC), hydroxypropylcellulose (HPC), hydroxypropylmethylcellulose (HPMC), hydroxyethylcellulose (HEC); polyvinylpyrrolidone (PVP), polyvinyl alcohol (PVA); and acrylic acid polymer, methacrylic acid copolymers, ethyl acrylate-methyl methacrylate copolymers, or a mixture thereof.

In another aspect, the method comprises administering the dosage form prepared as above.

In one aspect the invention provides a dosage form of mesalamine prepared according to the methods described herein.

In another aspect, the invention provides an article of manufacture comprising mesalamine prepared in accordance with the methods described herein and accompanying labeling and packaging to enable the article of manufacture to be shipped interstate.

In another aspect, a modified release mesalamine oral dosage form is provided comprising:
a) a therapeutically effective amount of mesalamine, ranging from about 200 mg to about 800 mg per dosage unit, formulated into one or more cores comprising said mesalamine and one or pharmaceutically acceptable excipients;
b) a release-modifying coat that substantially overlaps said core, wherein said coat comprises a mixture of a water-impermeable polymer and a water-swellable polymer;
c) wherein said dosage form releases said mesalamine in the following manner, when measured according to the USP.

The foregoing and other objects and aspects of the present invention are explained in detail in the detailed description and examples set forth herein.

### BRIEF DESCRIPTION OF THE FIGURES

FIG. 1 is a graphical representation of dissolution testing results of a mesalamine formulation prepared in accordance with Example 2 of the present invention. The dissolution was conducted at pH 1.2 as described in Example 9.

FIG. 2 is a graphical representation of dissolution testing results of a mesalamine formulation prepared in accordance with Example 2 of the present invention. The dissolution was conducted at pH 4.5 as described in Example 9.

FIG. 3 is a graphical representation of dissolution testing results of a mesalamine formulation prepared in accordance with Example 2 of the present invention. The dissolution was conducted at pH 6.8 as described in Example 9.

FIG. 4 is a graphical representation of dissolution testing results of a mesalamine formulation prepared in accordance with Example 2 of the present invention. The dissolution was conducted at pH 7.5 as described in Example 10.

FIG. 5 is a graphical representation of dissolution testing results of a mesalamine formulation prepared in accordance with Example 2 of the present invention. The dissolution was conducted at pH 1.2 for two hours followed by 6.8 as described in Example 11.

FIG. 6 is a graphical representation of dissolution testing results of a mesalamine formulation prepared in accordance with Example 2A of the present invention. The dissolution was conducted at pH 1.2 as described in Example 9.

FIG. 7 is a graphical representation of dissolution testing results of a mesalamine formulation prepared in accordance with 2A of the present invention. The dissolution was conducted at pH 6.8 as described in Example 9.

### DETAILED DESCRIPTION OF THE INVENTION

Definitions

In describing and claiming the present invention, the following terminology will be used in accordance with the definitions set forth below.

The singular forms “a,” “an,” and, “the” include plural referents unless the context clearly dictates otherwise. Thus, for example, reference to “a drug” includes reference to one or more of such drugs, and reference to “an excipient” includes reference to one or more of such excipients.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients.

As used herein, “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical, and medicinal arts.

As used herein, “mesalamine” refers to a compound known by the IUPAC name of 5-amino-2-hydroxybenzoic acid and having the structure: 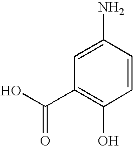
Mesalamine has a CAS Registry no. of 89-57-6, and is contained in the Merck Index as monograph no. 5931 (2005), which is incorporated herein by reference. The term “mesalamine compound” may also be used from time to time herein to refer to not only mesalamine, but also to encompass related compounds, such as analogs and homologs thereof, salts, such as acid addition salts thereof, prodrugs, isomers and metabolites thereof, as well as mixtures thereof as dictated by the context of its use. When referring to individual specific related compounds, or groups of compounds such as the acid addition salts, the specific technical name of each compound or molecule will be used, or the group will be specifically named, such as “mesalamine salts”.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, “blood level” may be used interchangeably with terms such as blood plasma concentration, plasma level, plasma concentration, serum level, serum concentration, serum blood level and serum blood concentration.

As used herein, “oral dosage form” and the like refers to a formulation that is ready for administration to a subject through the oral route of administration. Examples of known oral dosage forms, include without limitation, tablets, capsules, caplets, powders, pellets, granules, etc. Such formulations also include multilayered tablets wherein a given layer may represent a different drug. In some aspects, powders, pellets, and granules may be coated with a suitable polymer or a conventional coating material to achieve, for example, greater stability in the gastrointestinal tract, or to achieve the desired rate of release. Moreover, capsules containing a powder, pellets or granules may be further coated. Tablets and caplets may be scored to facilitate division of dosing. Alternatively, the dosage forms of the present invention may be unit dosage forms wherein the dosage form is intended to deliver one therapeutic dose per administration.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable carrier” and “carrier” may be used interchangeably, and refer to any inert and pharmaceutically acceptable material that has substantially no biological activity, and makes up a substantial part of the formulation.

The term “admixed” means that the drug and/or other ingredients can be dissolved, dispersed, or suspended in the carrier. In some cases, the drug may be uniformly admixed in the carrier.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

The term “modified release” as used herein refers to the drug release that is different from an immediate release. Typically, in an immediate release dosage form, about more than 80% of the drug is released from the dosage form in vitro within about 2 hrs. This release may be measured in terms of dissolution of the drug in the dissolution medium. In one aspect, the release is measured under USP conditions, i.e., where the pH is maintained at 1.2 for 2 hours, followed by a pH of 6.8 for the rest of the time. In another aspect, the release is measured at a pH of 1.2 for the entire period of measurement. Examples of such modified release include sustained release, slow-release, delayed-release, pulsatile release etc., which terms are generally known in the art and to the extent they mean a release other than an immediate release.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

The Invention

The present invention provides modified release mesalamine compound containing dosage forms with certain desirable in vitro dissolution properties and in vivo characteristics.

In one aspect, the invention provides methods for formulating a modified release mesalamine capsule dosage form. The capsule may contain one or more cores, depending on the dosage the capsule is intended to deliver, that comprise mesalamine and some excipients that are commonly known in the pharmaceutical industry. These cores are then coated with a specific mixture of polymers comprising a water-impermeable coating polymer and a water-swellable polymer. It has been discovered by the present inventors that this specific mixture of polymers provides the desired product with the desired in vitro and in vivo performance.

In one aspect, the cores may be prepared by the following process. Mesalamine and inert pharmaceutically acceptable excipients may be mixed thoroughly to achieve a substantially homogenous mixture. The excipients which may be employed are well known to those skilled in the art and include any conventional pharmaceutically acceptable tabletting excipients. Examples of suitable excipients include but are not limited to microcrystalline cellulose, dibasic calcium phosphate dihydrate, starch, sodium starch glycolate, crospovidone, croscarmellose sodium, magnesium stearate, lactose, maleic acid, colloidal silicon dioxide, talc, and glyceryl behenate.

The mixing of the excipients and mesalamine can be accomplished by using high shear granulators (mixers, blenders, etc). The homogenous mixture may be then processed into cores by a number of alternative processes such as granulation, spheronization, spheronization/extrusion, etc. These cores are then optionally dried. The drying process may provide certain advantages such content uniformity, ease of handling, etc.

Alternatively, the mesalamine and excipient mixture may be granulated with a water-impermeable polymeric dispersion to form granules of drug+excipient+water-impermeable polymer. The water impermeable polymer may be in one aspect ethylcellulose. The water impermeable polymer may be used at an amount ranging from about 1-20% in a non-aqueous solvent such as ethanol, isopropanol, or a mixture thereof. In some aspects, the water impermeable polymer amount may have the following ranges: from about 1-10%; from about 5-15%; from about 5-10%; from about 3-8%; from about 4-7% of the composition. In another aspect, the water impermeable polymer comprises about 6% of the composition. The amounts described herein are based on a w/w %.

This drug+excipient+water-impermeable polymer granulate is then optionally dried to substantially remove any residual solvents. Then the granulates may be optionally wetted to facilitate spheronization to extrude granules into an extruder. The operating conditions of the spheronization and extrusion processes and equipment are generally well-known in the art. The spheronization process yields cores that may be optionally sieved to optimize desired core size.

The cores thus obtained by either of the above alternate processes are then coated with a specific mixture of polymers comprising a water-impermeable coating polymer and a water-swellable polymer. The coating substantially completely surrounds the core. Examples of water-impermeable polymers include: ethyl cellulose, propyl cellulose, etc. Examples of water-swellable polymers include: hydroxypropylmethylcellulose, gums, alginates, etc.

In one aspect, the coating mixture comprises HPMC and ethylcellulose dispersed in an aqueous or substantially nonaqueous solvent. A substantially nonaqueous solvent may be selected form a variety of solvents such as methanol, ethanol, isopropanol, acetone, or a mixture thereof. The HPMC and ethylcellulose may be selected from one of several grades that are commercially available, as described elsewhere in this application.

The amount of water-insoluble polymer in the coating may range from about 0.5% to about 10% of the modified release formulation. In some aspects, the amount of water-insoluble polymer in the coating may range as following: from about 1-10%; from about 2-8%; from about 2-6%; from about 1-5%; from about 1-3%; from about 2-3% of the modified release composition. In some specific aspects, the water-insoluble polymer in the coating may amount to about 2.5% of the modified release composition. These amounts are expressed as w/w %.

The amount of water-swellable polymer in the coating may range from about 0.1% to about 5% of the modified release formulation. In some aspects, the amount of water-swellable polymer in the coating may range as following: from about 0.5% to about 3%; from about 0.5% to about 2%; from about 0.5% to about 1.5% of the modified release composition. In some specific aspects, the water-swellable polymer in the coating may amount to about 1% of the modified release composition. These amounts are expressed as w/w %.

In one aspect, the ratio of water-insoluble polymer to the water-swellable polymer may be from about 80 to about 20. In another aspect, that ratio may be: from about 70 to about 30; from about 60 to about 40; from about 50 to about 50; from about 40 to about 60; from about 30 to 70; from about 20 to about 80.

The polymeric coating layer may be accomplished by directly applying the coating polymer mixture alone or together with a binder, either as a solution or as a powder. For example, the binder may be provided as a solution or as a dispersion and may be applied just prior to, or together with the polymer mixture. The polymer mixture may be applied as a dispersion (which may be a solution, suspension or as an emulsion) if the binder is provided as a solution or as a powder. Alternatively, the binder may be provided as a fine powder and the polymer mixture may be provided as a dispersion. Upon contact with the polymer dispersion, the binder powder may become a solution or suspension which then forms a binding film on the cores and thus facilitate the coating of the polymer onto the cores.

The polymeric coating layer may be applied to the core according to methods generally known in the art. For example, a two-step process, within which the steps may be repeated a sufficient number of times as necessary to build the thickness of the polymeric coating layer to achieve the desired in vitro and in vivo characteristics. In the first step, the core is wet with the binder dispersion which serves to adhere the powdered polymeric coating particles to the wet core. Suitable binder dispersions may include conventional pharmaceutically acceptable binder agents solubilized in a suitable solvent. Specific examples of binder agents include but are not limited to vinyl polymers, such as polyvinylpyrrolidone, polyvinyl alcohol, and the like; cellulosic polymers, such as HPMC, HEC, HPC, and the like; acrylic polymers and copolymers such as methacrylic acid copolymers, ethyl acrylate-methylmethacrylate copolymers, and the like; natural or synthetic gums, such as guar gum, arabic gum, xanthan gum, and the like; proteins or carbohydrates, such as gelatin, pectin, and the like; and mixtures thereof. In some aspects, polyvinylpyrrolidone is the preferred binder agent.

Suitable solvents for solubilizing the binder agents include solvents which are capable of substantially completely solubilizing the specific binder agent(s) selected and which are pharmaceutically and biologically acceptable for ingestion. Suitable solvents will be readily determinable by those skilled in the art. Water is currently the preferred solvent for solubilizing the binder agent. However, other examples of suitable solvents will be appreciated by those skilled in the art and are contemplated by the methods of the present invention.

The binder solution should be of sufficient viscosity to enable the wetting of the cores by any suitable wetting technique known to those skilled in the art. For example, the cores may be wetted with the binder solution by rotating the cores in a bath containing the binder solution. The cores may be suitably wetted by manual application of the binder dispersion by layer the binder solution over the cores as the cores are rotating in a conventional coating pan. Alternatively, the cores may be wetted by spraying the binder dispersion on the cores. In one aspect, the wetting step is advantageously carried out using conventional automated pan coating equipment wherein the cores are sprayed with the binder dispersion while rotating in the pan.

To provide the coating layer, the wetted cores may be coated with dry, powdered polymeric coating particles which adhere to the binder-wetted core due to the presence of the binder on the surface of the core.

The polymeric coating mixture may be comprised of any suitable water-impermeable and water-swellable polymers known to those skilled in the art. For example, suitable polymers include: cellulosic polymers, such as methylcellulose (MC), carboxymethylcellulose (CMC), hydroxypropylcellulose (HPC), hydroxylpropylmethylcellulose (HPMC), hydroxyethylcellulose (HEC), and the like; vinyl polymers, such as polyvinylpyrrolidone (PVP), polyvinyl alcohol (PVA), and the like; acrylic polymers and copolymers, such as acrylic acid polymer, methacrylic acid copolymers, ethyl acrylate-methyl methacrylate copolymers, and the like; and mixtures thereof. Currently, the preferred polymers include ethylcellulose and HPMC.

In one aspect, the amount of polymers in the polymeric coating mixture may range from about 0.5% to about 10% of the dispersion. In some aspects, the range may be as following: from about 1-10%; from about 2-8%; from about 2-6%; from about 1-5%; from about 3-5%; from about 4-5%. In some specific aspects, the polymers in the polymeric coating mixture comprise about 4.5%. The amounts described herein are w/w %.

HPMC may comprise material of certain viscosity and molecular weight or alternately may comprise mixtures or blends of two or more different forms of HPMC. In one aspect, the mixture may comprise of HPMC having differing molecular weights and solubility characteristics. For example, the mixture may comprise of: a) HPMC having i) a typical weight percent substitution corresponding to about 30% methoxyl and about 10% hydroxypropoxyl groups, and ii) a nominal viscosity of about 2% watery solution at 20 C ranging from about 5 to about 100 mPa.s e.g., METHOCEL E5; and b) HPMC having i) a typical weight percent substitution corresponding to about 20% methyoxyl and about 8% hydroxypropoxyl groups, and ii) a nominal viscosity of about 2% watery solution at 20 C ranging from about 4,000 to about 100,000 mPa.s (e.g., METHOCEL K15M).

Because the formulations and methods of the present invention may include either a single HPMC or a blend of two or more different forms of HPMC as the coating, for simplicity, the term HPMC as used herein, including the claims, refers to either a single HPMC or a blend of two or more forms of the polymer.

Alternatively, the swellable polymeric coating layer may be comprising of other substances which are functional equivalents to HPMC. For example, polysaccharides, such as gelatin, saccharose, sorbitol, mannanes, and jaluronic acid; polyaminoacids; polyalcohols; polyglycols may also work.

In addition to the foregoing, the polymeric coating layer may also include other excipients such as lubricants, flow promoting agents, plasticizers, antisticking agents, natural and synthetic flavorings and natural and synthetic colorants. Specific examples of additional excipients include polyethylene glycol, polyvinylpyrrolidone, talc, magnesium stearate, glyceryl behenate, stearic acid, and titanium dioxide.

After the powdered polymeric coating layer is applied to the core, the process may be repeated one or more additional times in order to build the thickness of the polymeric coating layer around the core. The number of repetitions is dependent upon the desired predetermined in vitro dissolution profile and in vivo performance. A sufficient number of coating cycles are performed so as to produce a core:coating layer weight ratio of between about 40:1 and about 1:5 inclusive, or a thickness in excess of about 10 uM, and up to about 500 uM. In one aspect, a sufficient number of coating cycles are completed so as to produce a core:coating layer weight ratio of between about 5:1 and about 1:3 inclusive, or a thickness of about 50 uM and about 200-400 uM.

The present invention also provides modified release formulations of mesalamine that are suitable for oral administration and delivery in the gastro-intestinal tract. A typical formulation includes: (a) a core comprising mesalamine, and (b) a polymeric coating layer substantially surrounding the core comprising a mixture of water-impermeable polymer and a water-swellable polymer. As described hereinabove, in one aspect, the polymeric coating layer is applied with or without a binder solution or dispersion. The coating cycle may be repeated one or more times to obtain the necessary coating thickness and other criteria to provide the desired in vitro and in vivo characteristics.

If desired, the formulations of the present invention may be provided in the form of capsules wherein the core of the present invention is used to fill in a conventional hard or soft-gelatin capsule. Encapsulation within a soft-gelatin capsule is also achievable with conventional techniques.

Additionally, the present invention also provides methods of achieving desired therapeutic benefit from mesalamine therapy by administering to the patient the oral dosage form prepared according to the presently disclosed methods. Suitable patient populations for which the methods of the present invention are directed include mammals in general, and in particular, humans.

### EXAMPLES

The following examples are provided to illustrate the present invention, and should not be construed as limiting thereof. All percentages are in percent by weight of the tablet unless otherwise indicated. Disintegration tests are carried out according to the standard procedures set forth in the United States Pharmacopoeia for testing the disintegration of tablets.

### Example 1

### Granulation

Pass Mesalamine through a ASTM #30 mesh. Mix Mesalamine (500 mg) and Talc (10 mg). Dissolve ethylcellulose in a sufficient amount of Isopropyl alcohol to make 4% solution. Drug load Mesalamine onto non pareil sugar beads (139.18 mg) with ethylcellulose (75.45 mg) solution. Sugar beads of size #25-30 or #30-35 may be used for this purpose. Drug loading can be done in a rotogranulator with tangential coating or a conventional coating pan with powder spraying/layering or a similar equipment. Film coat these beads with a solution of Ethyl cellulose (19.02 mg) and HPMC (17.21 mg) in methyl alcohol with castor oil (5.43 mg) as plasticizer in a conventional coating pan. Fill the capsule size “00” elongated with sufficient amount of beads so that the total Mesalamine content is 500 mg.

### Example 1A

### Granulation

Pass Mesalamine through a ASTM #30 mesh. Mix Mesalamine (500 mg) and Talc (10 mg). Dissolve ethylcellulose in a sufficient amount of Isopropyl alcohol to make 2.75% solution. Drug load Mesalamine onto non pareil sugar beads (139.18 mg) with ethylcellulose (75.45 mg) solution. Sugar beads of size #25-30 or #30-35 may be used for this purpose. Drug loading can be done in a rotogranulator with tangential coating or a conventional coating pan with powder spraying/layering or a similar equipment. Film coat these beads with a solution of Ethyl cellulose (22.83 mg) and HPMC (20.65 mg) in methyl alcohol with castor oil (6.52 mg) as plasticizer in a conventional coating pan. Fill the capsule size “00” elongated with sufficient amount of beads so that the total Mesalamine content is 500 mg.

### Example 2

### Fluid Bed Coating

The mesalamine containing cores are prepared as in Example No. 1. The cores containing 500 mg of Mesalamine are coated with the ingredients as in Example 1 using a fluid bed apparatus. A Glatt GPCG 3.1 can be used for this purpose. Fill the capsule size “00” elongated with sufficient amount of beads so that the total Mesalamine content is 500 mg.

### Example 2A

### Fluid Bed Coating

The mesalamine containing cores are prepared as in Example No. 1A. The cores containing 500 mg of Mesalamine are coated with the ingredients as in Example 1 using a fluid bed apparatus. A Glatt GPCG 3.1 can be used for this purpose. Fill the capsule size “00” elongated with sufficient amount of beads so that the total Mesalamine content is 500 mg.

### Example 3

Mesalamine 200 mg is used per dosage form which may be prepared similar to Example 1A except for the difference in dosage amount and the corresponding differences in the inactive ingredients.

### Example 4

Mesalamine 250 mg is used per dosage form which may be prepared similar to example 1A except for the difference in dosage amount and the corresponding differences in the inactive ingredients.

### Example 5

Mesalamine 300 mg is used per dosage form which may be prepared similar to example 1A except for the difference in dosage amount and the corresponding differences in the inactive ingredients.

### Example 6

Mesalamine 400 mg is used per dosage form which may be prepared similar to example 1A except for the difference in dosage amount and the corresponding differences in the inactive ingredients.

### Example 7

Mesalamine 600 mg is used per dosage form which may be prepared similar to example 1A except for the difference in dosage amount and the corresponding differences in the inactive ingredients.

### Example 8

Mesalamine 800 mg is used per dosage form which may be prepared similar to example 1A except for the difference in dosage amount and the corresponding differences in the inactive ingredients.

### Example 9

To validate the robustness of the present invention in terms of coating composition, coating methodology and commercial feasibility, in vitro dissolution tests in so-called “discriminating media” under different pH values were conducted. The details of these experiments are shown below in Table 1 for dosage forms presented in Examples 1A and 2A.

TABLE 1

| | BUFFER | | SPEED | | | PATH- | |
|---|---|---|---|---|---|---|---|
| NO. | CONCENTRATION | VOLUME | (RPM) | TEMP | APPARATUS | LENGTH | WAVELENGTH |
| 1 | pH 1.2 SGF w/o | 1000 | 100 | 37 ± 0.5° C. | 2, paddles | 0.1 | 303 |
| | pepsin | | | | | | |
| 2 | pH4.5 Phosphate | 1000 | 100 | 37 ± 0.5° C. | 2, paddles | 0.1 | 298 |
| 3 | pH 6.8 Phosphate | 1000 | 100 | 37 ± 0.5° C. | 2, paddles | 0.1 | 330 |
| | Buffer | | | | | | |

Sampling points: - 0.5, 1, 2, 3, 4, 6, 8 hrs

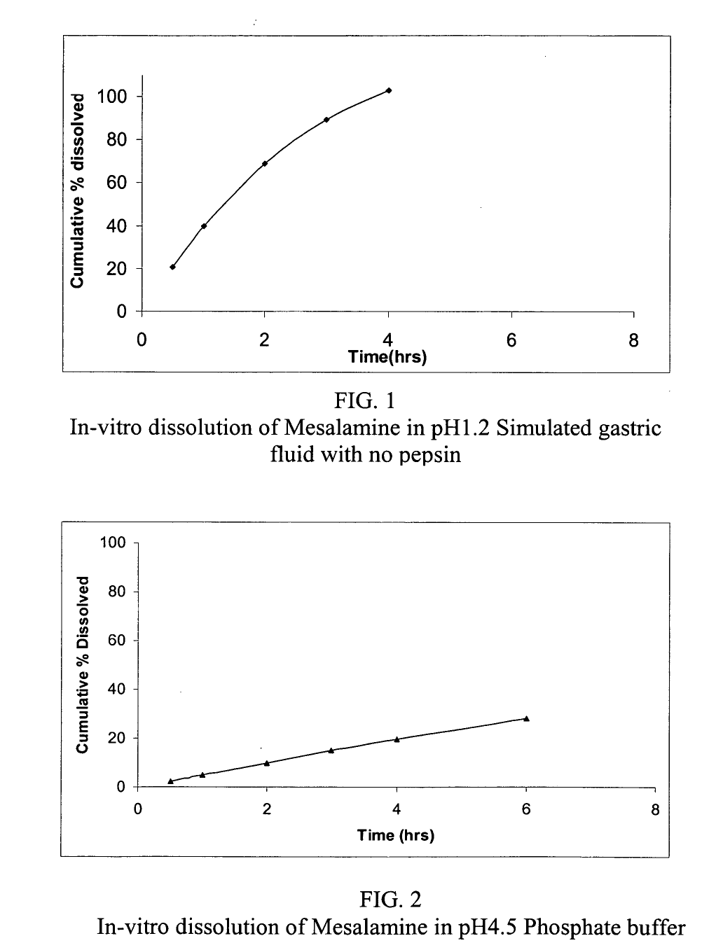

The resulting data are presented as Tables 2-4, and in graphical form as FIGS. 1-3 (for pH 1.2, pH 4.5, and pH 6.8, respectively) for the product from Example 1A. The data confirm not only the process validation. Further, surprisingly, the data also show comparability of the present formulations to the branded Pentasa formulations. This equivalency is robust, and is reproducible in discriminating media among various pH values. This result is quite unexpected and surprising yet highly desirable.

TABLE 2
pH 1.2 Simulated Gastric Fluid w/o pepsin

| Time | Mesalamine, Formulation 1A |
|---|---|
| (hrs) | Cumulative % released |
| 0.5 | 20.55 |
| 1 | 39.82 |
| 2 | 69.14 |
| 3 | 89.42 |
| 4 | 102.66 |
| 6 | SNT* |
| 8 | SNT* |

*Sample not taken

A graphical representation of these results is shown in FIG. 1.

TABLE 3
pH 4.5 Phosphate Buffer

| Time | Mesalamine, Formulation 1A |
|---|---|
| (hrs) | Cumulative % released |
| 0.5 | 2.33 |
| 1 | 4.94 |
| 2 | 10.00 |
| 3 | 14.92 |
| 4 | 19.61 |
| 6 | 28.33 |
| 8 | SNT* |

*Sample not taken

A graphical representation of these results is shown in FIG. 2.

TABLE 4
pH 6.8 Simulated Intestinal Fluid w/o pancreatin (Phosphate Buffer)

| Time | Mesalamine, Formulation 1A |
|---|---|
| (hrs) | Cumulative % released |
| 0.5 | 5.90 |
| 1 | 12.75 |
| 2 | 26.00 |
| 3 | 38.25 |
| 4 | 49.45 |
| 6 | 67.90 |
| 8 | 81.35 |

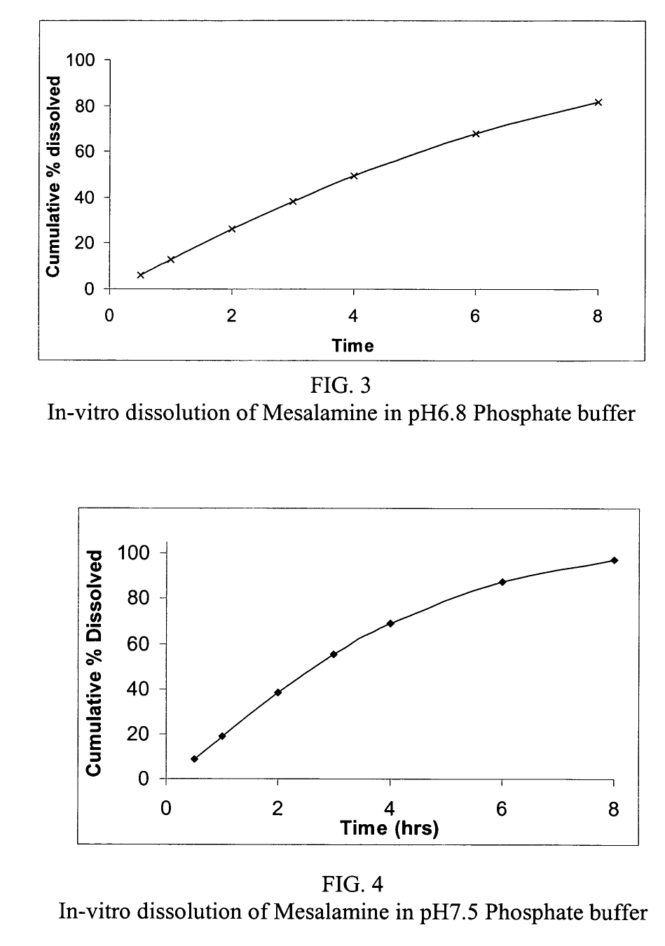

A graphical representation of these results is shown in FIG. 3.

### Example 10

Dissolution testing was conducted according to the official methodology in United States pharmacopeia 27, monograph titled “Mesalamine Extended Release Capsules,” which is incorporated by reference. Briefly, for each test, either one capsule of branded product, Pentasa or one capsule of the present invention (designated as CPI) with 500 mg of equivalent active cores was used. The pH of the medium was maintained at 7.5 with phosphate buffer. 8 mL samples were withdrawn at predetermined times using an automated sampler. The Mesalamine concentration in each sample was determined using an UV-Vis spectrophotometer at wavelength of 330 nm. The percentage of Mesalamine released over time was calculated and plotted as an average of 6 runs using calibration curves consistent with Beer's law.

Further experimental details are provided as following in Table 5.

TABLE 5

| BUFFER | | SPEED | | | PATH- | |
|---|---|---|---|---|---|---|
| CONCENTRATION | VOLUME | (RPM) | TEMP | APPARATUS | LENGTH | WAVELENGTH |
| USP | 1000 | 100 | 37 ± 0.5 C. | 2, paddles | 0.1 | 330 |
| CONDITIONS | | | | | | |
| pH 7.5 | | | | | | |
| phosphate | | | | | | |
| buffer | | | | | | |

Sampling points: - 0.5, 1, 2, 3, 4, 6, 8 hrs

The resulting dissolution data are presented in Table 6 and in graphical form as FIG. 4 for the product of Example 1A. The data indicate that the mesalamine modified dosage form as formulated and prepared according to the present invention has met the Official USP dissolution requirements.

TABLE 6
pH 7.5 Phosphate buffer (USP Media)

| Time | Mesalamine, Formulation 1A |
|---|---|
| (hrs) | Cumulative % released |
| 0.5 | 8.68 |
| 1 | 18.91 |
| 2 | 38.43 |
| 3 | 55.18 |
| 4 | 68.96 |
| 6 | 87.4 |
| 8 | 97.04 |

A graphical representation of these results is shown in FIG. 4.

### Example 11

Another indication for the robustness of the formulation as well as for in vivo performance is a measure of the release of the product with the pH of the medium varying over the course of the experiment. Dissolution testing was conducted similar to the official methodology in United States Pharmacopeia 27, as in the monograph titled “Delayed-Release (Enteric coated) Articles—General Drug Release Standard”, method A, but using Simulated gastric Fluid without the enzyme Pepsin (pH 1.2) instead of 0.1N Hydrochloric acid. Briefly, for each test, either one capsule of branded product, Pentasa or one capsule of the present invention (designated as CPI) with 500 mg of equivalent active cores was used. 8 mL samples were withdrawn at predetermined times using an automated sampler. The Mesalamine concentration in each sample was determined using an UV-Vis spectrophotometer. Wavelength of 303 was used for the SGF pH 1.2 and wavelength of 330 nm was used for pH 6.8 dissolution media. The percentage of Mesalamine released over time was calculated and plotted as an average of 6 runs using calibration curves consistent with Beer's law.

Further experimental details are provided as following in Table 7.

TABLE 7

| BUFFER | | SPEED | | | PATH- | |
|---|---|---|---|---|---|---|
| CONCENTRATION | VOLUME | (RPM) | TEMP | APPARATUS | LENGTH | WAVELENGTH |
| pH 1.2 SGF- | 1000 | 100 | 37 ± 0.5 C. | 2, | 0.1 cm | 303 |
| 2 hrs | | | | PADDLES | | 330 |
| pH 6.8 - 6 hrs | | | | | | |

Sampling points: 0.5, 1, 2, 3, 4, 6, 8 hrs.

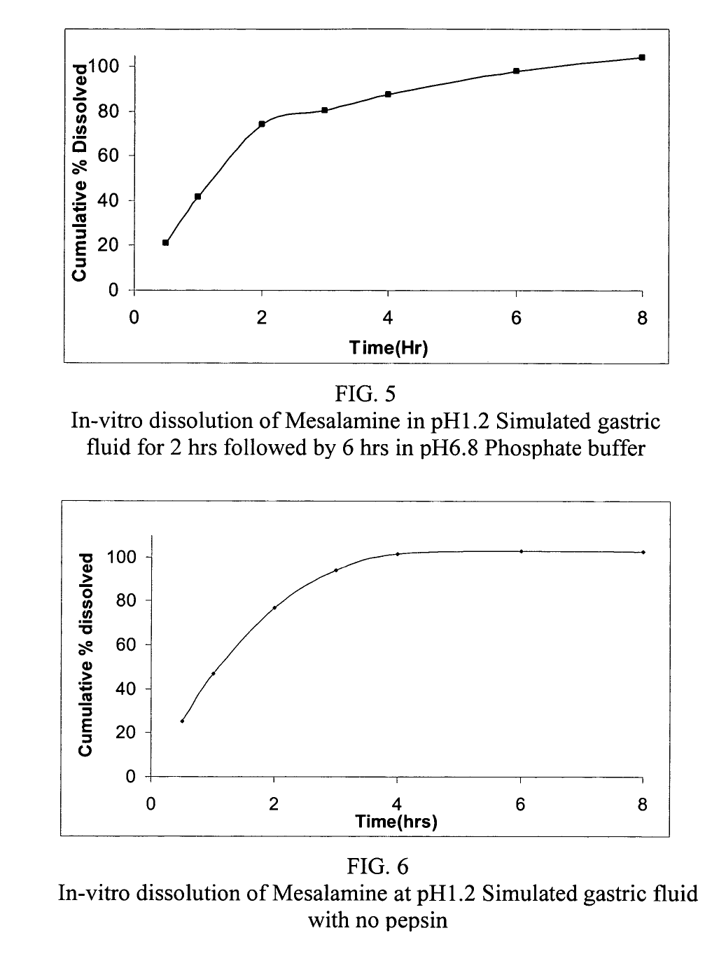

The resulting dissolution data are presented as Table 8, and in graphical form as FIG. 5 for the product of Example 1A. The data indicate that the mesalamine modified dosage form as formulated and prepared according to the present invention has acceptable dissolution profile under the varying pH conditions. This result is also found to be quite comparable to the dissolution profile of the branded product, Pentasa, under similar in vitro dissolution conditions. This is also quite an unexpected result.

TABLE 8
2 hours in pH 1.2 Simulated gastric fluid followed by remaining
6 hours in pH 6.8 Phosphate buffer (USP Media)

| Time | Mesalamine, Formulation 1A |
|---|---|
| (hrs) | Cumulative % released |
| 0.5 | 20.96 |
| 1 | 41.70 |
| 2 | 74.15 |
| 3 | 80.30 |
| 4 | 87.59 |
| 6 | 97.93 |
| 8 | 104.00 |

A graphical representation of these results is shown in FIG. 5.

### Example 12

Yet another batch of the product was subjected to dissolution tests as described above. Product samples of the present invention as prepared according to Example 2A were used here.

These sample products were subjected to in-vitro dissolution testing under various conditions, as given in Table 1 above. The specific dissolution conditions were as described in Example 9 and results are outlined in the tables below.

TABLE 9
pH 1.2 Simulated Gastric Fluid w/o pepsin

| Time | Mesalamine, Formulation 2A |
|---|---|
| (hrs) | Cumulative % released |
| 0.5 | 25.36 |
| 1 | 46.91 |
| 2 | 76.8 |
| 3 | 93.85 |
| 4 | 101.49 |
| 6 | 103.06 |
| 8 | 102.61 |

*Sample not taken

A graphical representation of these results is shown in FIG. 6.

TABLE 11
pH 6.8 Simulated Intestinal Fluid w/o pancreatin (Phosphate Buffer)

| Time | Mesalamine, Formulation 2A |
|---|---|
| (hrs) | Cumulative % released |
| 0.5 | 8.68 |
| 1 | 18.09 |
| 2 | 34.81 |
| 3 | 48.92 |
| 4 | 60.65 |
| 6 | 78.54 |
| 8 | 90.28 |

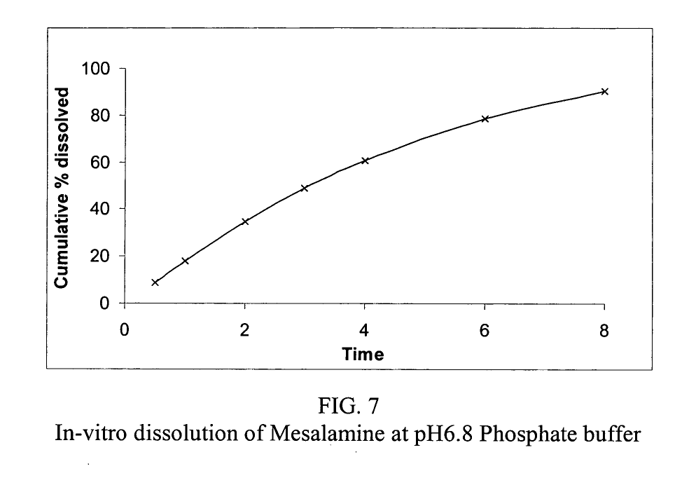

A graphical representation of these results is shown in FIG. 7.

The foregoing is illustrative of the present invention and is not to be construed as limiting thereof. The invention is defined by the following claims, with equivalents of the claims to be included therein.

What is claimed is:

1. A modified release mesalamine oral dosage form comprising:
a) a therapeutically effective amount of mesalamine, ranging from about 200 mg to about 800 mg per dosage unit, formulated into one or more cores comprising said mesalamine and one or pharmaceutically acceptable excipients;
b) a release-modifying coat that substantially or completely overlaps said core, wherein said coat comprises a mixture of a water-impermeable polymer and a water-swellable polymer;
wherein, the dosage form provides a dissolution profile selected from the group consisting of:
i) about 15% to about 25% of the drug is released by 60 minutes; about 35% to about 45% of the drug is released by 2 hrs; about 70% to about 85% of the drug is released by 4 hrs; and about 95% to about 105% of the drug is released by 8 hrs when dissolution test is performed using pH 7.5 phosphate buffer;
ii) about 15% or less of the drug is released by 60 minutes; about 20% to about 35% of the drug is released by 2 hrs; about 40% to about 60% of the drug is released by 4 hrs; and about 75% to about 90% of the drug is released by 8 hrs when dissolution test is performed using pH 6.8 phosphate buffer and simulated intestinal fluid without pancreatin;
iii) about 20% to about 45% of the drug is released by 60 minutes; about 35% to about 75% of the drug is released by 2 hrs; about 90% to about 100% of the drug is released by 4 hrs, when dissolution test is performed using pH 1.2 simulated gastric fluid without pepsin; and
iv) about 3% to about 6% of the drug is released by 60 minutes; about 8% to about 12% of the drug is released by 2 hrs; about 16% to about 20% of the drug is released by 4 hrs; and more than about 25% the drug is released by 8 hrs when dissolution test is performed using pH 4.5 phosphate buffer.

2. The composition according to claim 1, wherein the one or more pharmaceutically acceptable exicipients are selected from the group consisting of: microcrystalline cellulose, dibasic calcium phosphate dihydrate, starch, sodium starch glycolate, crospovidone, croscarmellose sodium, magnesium stearate, lactose, maleic acid, colloidal silicon dioxide, talc, and glyceryl behenate, or a mixture thereof.

3. The composition according to claim 1, wherein the water-impermeable polymer comprises from about 1% to about 10% w/w of the composition, and is selected from the group consisting of ethylcellulose, propylcellulose, isopropylcellulose, or a mixture thereof.

4. The composition according to claim 1, wherein the water-swellable polymer comprises from about 1% to about 10% w/w of the composition, and is selected from the group consisting of methylcellulose (MC), carboxymethylcellulose (CMC), hydroxypropylcellulose (HPC), hydroxypropylmethylcellulose (HPMC), hydroxyethylcellulose (HEC); polyvinylpyrrolidone (PVP), polyvinyl alcohol (PVA); and acrylic acid polymer, methacrylic acid copolymers, ethyl acrylate-methyl methacrylate copolymers, or a mixture thereof.

5. The composition of claim 3, wherein the water-impermeable polymer is ethylcellulose and comprises from about 1% to about 10% w/w of the composition, and the water-swellable polymer is hydroxymethylpropylcellulose and comprises from about 1% to about 10% w/w of the composition.

6. A method of preparing a modified release mesalamine oral dosage form comprising the steps of:
a) providing an inert core of substantially uniform size;
b) providing mesalamine dispersion and optionally a binder dispersion;
c) layering said core with mesalamine dispersion simultaneously with or after optional layering of said core with binder dispersion to provide mesalamine core;
d) preparing a dispersion of a water-impermeable polymer and a water-swellable polymer to produce a coating polymer dispersion;
e) coating said mesalamine core with said coating polymer dispersion to obtain coated mesalamine core; and
f) providing modified release mesalamine capsules by filling empty capsules with one or more coated mesalamine cores.

7. A modified release mesalamine oral dosage form comprising:
a) a therapeutically effective amount of mesalamine, ranging from about 200 mg to about 800 mg per dosage unit, formulated into one or more cores comprising said mesalamine and one or pharmaceutically acceptable excipients;
b) a release-modifying coat that substantially completely overlaps said core, wherein said coat comprises a mixture of a water-impermeable polymer and a water-swellable polymer;
wherein, the dosage form provides a dissolution profile selected from the group consisting of:
i) about 15% to about 25% of the drug is released by 60 minutes; about 35% to about 45% of the drug is released by 2 hrs; about 70% to about 85% of the drug is released by 4 hrs; and about 95% to about 105% of the drug is released by 8 hrs when dissolution test is performed using pH 7.5 phosphate buffer;
ii) about 15% or less of the drug is released by 60 minutes; about 20% to about 35% of the drug is released by 2 hrs; about 40% to about 60% of the drug is released by 4 hrs; and about 75% to about 90% of the drug is released by 8 hrs when dissolution test is performed using pH 6.8 phosphate buffer and simulated intestinal fluid without pancreatin;
iii) about 20% to about 45% of the drug is released by 60 minutes; about 35% to about 75% of the drug is released by 2 hrs; about 90% to about 100% of the drug is released by 4 hrs, when dissolution test is performed using pH 1.2 simulated gastric fluid without pepsin; and
iv) about 3% to about 6% of the drug is released by 60 minutes; about 8% to about 12% of the drug is released by 2 hrs; about 16% to about 20% of the drug is released by 4 hrs; and more than about 25% the drug is released by 8 hrs when dissolution test is performed using pH 4.5 phosphate buffer;
wherein said release-modifying coating composition comprises from about 1% to about 10% ethylcellulose and from about 1% to about 10% hydroxypropylmethylcellulose; and wherein said mesalamine core comprises an inert bead.

8. The method of claim 6, wherein the one or more pharmaceutically acceptable exicipients are selected from the group consisting of: microcrystalline cellulose, dibasic calcium phosphate dihydrate, starch, sodium starch glycolate, crospovidone, croscarmellose sodium, magnesium stearate, lactose, maleic acid, colloidal silicon dioxide, talc, and glyceryl behenate, or a mixture thereof.

9. The method of claim 6, wherein the water-impermeable polymer comprises from about 1% to about 10% of the composition, and is selected from the group consisting of ethylcellulose, propylcellulose, isopropylcellulose, or a mixture thereof.

10. The method of claim 6, wherein the water-swellable polymer comprises from about 1% to about 10% of the composition, and is selected from the group consisting of methylcellulose (MC), carboxymethylcellulose (CMC), hydroxypropylcellulose (HPC), hydroxypropylmethylcellulose (HPMC), hydroxyethylcellulose (HEC); polyvinylpyrrolidone (PVP), polyvinyl alcohol (PVA); and acrylic acid polymer, methacrylic acid copolymers, ethyl acrylate-methyl methacrylate copolymers, or a mixture thereof.
